(19) United States
(12) Patent Application Publication
(10) Pub. No.: US 2006/0079723 A1
(43) Pub. Date: Apr. 13, 2006

(54) Catalyst and process for the conversion of oxygenates to olefins

(76) Inventors: Machteld M. Mertens, Boortmeerbeek (BE); Marcel J. Janssen, Kessel-Lo (BE); Luc R.M. Martens, Meise (BE); Kenneth Ray Clem, Humble, TX (US)

Correspondence Address: EXXONMOBIL CHEMICAL COMPANY, 5200 BAYWAY DRIVE, P.O. BOX 2149, BAYTOWN, TX, 77522-2149, US

(21) Appl. No.: 11/198,655

(22) Filed: Aug. 5, 2005

Related U.S. Application Data
(63) Continuation-in-part of application No. 10/964,172, filed on Oct. 12, 2004.

(51) Int. Cl. B01J 27/182; C07C 1/00

(52) U.S. Cl. 585/639; 502/214

(57) ABSTRACT

A catalyst composition for use in the conversion of oxygenates to olefins comprises a first molecular sieve comprising a CHA framework type material and a second molecular sieve comprising an AFI framework type material, wherein said second molecular sieve is present in an amount up to 0.75% by weight of said first molecular sieve.

### CROSS REFERENCE TO RELATED APPLICATIONS

This is a continuation-in-part application of U.S. application Ser. No. 10/964,172 filed Oct. 13, 2004, the disclosure of which is fully incorporated herein by reference.

### FIELD OF INVENTION

This invention relates to a catalyst and process for the conversion of oxygenates, particularly methanol, to olefins, particularly ethylene and propylene.

### BACKGROUND OF INVENTION

Light olefins, such as ethylene, propylene, butylenes and mixtures thereof, serve as feeds for the production of numerous important chemicals and polymers. Typically, C₂-C₄ light olefins are produced by cracking petroleum refinery streams, such as C₃+ paraffinic feeds. In view of limited supply of competitive petroleum feeds, production of low cost light olefins from petroleum feeds is subject to waning supplies. Efforts to develop light olefin production technologies based on alternative feeds have therefore increased.

An important type of alternative feed for the production of light olefins is oxygenates, such as C₁-C₄ alkanols, especially methanol and ethanol; C₂-C₄ dialkyl ethers, especially dimethyl ether (DME), methyl ethyl ether and diethyl ether; dimethyl carbonate and methyl formate, and mixtures thereof. Many of these oxygenates may be produced from alternative sources by fermentation, or from synthesis gas derived from natural gas, petroleum liquids, carbonaceous materials, including coal, recycled plastic, municipal waste, or any organic material. Because of the wide variety of sources, alcohol, alcohol derivatives, and other oxygenates have promise as an economical, non-petroleum sources for light olefin production.

The preferred process for converting an oxygenate feedstock, such as methanol, into one or more olefin(s), primarily ethylene and/or propylene, involves contacting the feedstock with a crystalline molecular sieve catalyst composition. Crystalline molecular sieves have a 3-dimensional, four-connected framework structure of corner-sharing [TO₄] tetrahedra, where T is any tetrahedrally coordinated cation. Among the known forms of molecular sieve are aluminosilicates, which contain a three-dimensional microporous crystal framework structure of [SiO₄] and [AlO₄] corner sharing tetrahedral units silicoaluminophosphates (SAPOs), in which the framework structure is composed of [SiO₄], [AlO₄] and [PO₄] corner sharing tetrahedral units.

Molecular sieves have been classified by the Structure Commission of the International Zeolite Association according to the rules of the IUPAC Commission on Zeolite Nomenclature. According to this classification, framework-type zeolite and zeolite-type molecular sieves, for which a structure has been established, are assigned a three letter code and are described in the Atlas of Zeolite Framework Types, 5th edition, Elsevier, London, England (2001), which is herein fully incorporated by reference.

Among the molecular sieves that have been investigated for use as oxygenate conversion catalysts, materials having the framework type of the zeolitic mineral chabazite (CHA) have shown particular promise. For example, SAPO-34 is a crystalline silicoaluminophosphate molecular sieve of the CHA framework type and has been found to exhibit relatively high product selectivity to ethylene and propylene, and low product selectivity to paraffins and olefins with four or more carbon atoms.

The preparation and characterization of SAPO-34 have been reported in several publications, including U.S. Pat. No. 4,440,871; J. Chen et al. in “Studies in Surface Science and Catalysis”, Vol. 84, pp. 1731-1738; U.S. Pat. No. 5,279,810; J. Chen et al. in “Journal of Physical Chemistry”, Vol. 98, pp. 10216-10224 (1994); J. Chen et al. in “Catalysis Letters”, Vol. 28, pp. 241-248 (1994); A. M. Prakash et al. in “Journal of the Chemical Society, Faraday Transactions” Vol. 90(15), pp. 2291-2296 (1994); Yan Xu et al. in “Journal of the Chemical Society, Faraday Transactions” Vol. 86(2), pp. 425-429 (1990).

Regular crystalline molecular sieves, such as the CHA framework type materials, are built from structurally invariant building units, called Periodic Building Units, and are periodically ordered in three dimensions. Disordered structures showing periodic ordering in less than three dimensions are, however, also known. One such disordered structure is a disordered planar intergrowth in which the building units from more than one framework type, e.g., both AEI and CHA, are present. One well-known method for characterizing crystalline materials with planar faults is DIFFaX, a computer program based on a mathematical model for calculating intensities from crystals containing planar faults (see M. M. J. Tracey et al., Proceedings of the Royal Chemical Society, London, A [1991], Vol. 433, pp. 499-520).

International Patent Publication No. WO 02/70407, published Sep. 12, 2002 and incorporated herein by reference, discloses a silicoaluminophosphate molecular sieve, now designated EMM-2, comprising at least one intergrown form of molecular sieves having AEI and CHA framework types, wherein said intergrown form has an AEI/CHA ratio of from about 5/95 to 40/60 as determined by DIFFaX analysis, using the powder X-ray diffraction pattern of a calcined sample of said silicoaluminophosphate molecular sieve. EMM-2 is shown to be active as a catalyst in the production of light olefins from methanol (MTO).

U.S. Pat. No. 6,334,994, incorporated herein by reference, discloses a silicoaluminophosphate molecular sieve, referred to as RUW-19, which is also said to be an AEI/CHA mixed phase composition. In particular, RUW-19 is reported as having peaks characteristic of both AEI and CHA structure type molecular sieves, except that the broad feature centered at about 16.9 (2θ) in RUW-19 replaces the pair of reflections centered at about 17.0 (2θ) in AEI materials and RUW-19 does not have the reflections associated with CHA materials centered at 2θ values of 17.8 and 24.8. DIFFaX analysis of the X-ray diffraction pattern of RUW-19 as produced in Examples 1, 2 and 3 of U.S. Pat. No. 6,334,994 indicates that these materials are characterized by single intergrown forms of AEI and CHA structure type molecular sieves with AEI/CHA ratios of about 60/40, 65/35 and 70/30. RUW-19 is reported to be active as a catalyst in the production of light olefins from methanol (MTO).

It is known that the synthesis of CHA framework type materials, including AEI/CHA intergrowths, frequently produces impurity phases and, in particular, that the production of AFI framework type materials, such as SAPO-5, often competes with that of CHA framework type materials. For example, the RUW-19 material obtained in Examples 2 and 3 of U.S. Pat. No. 6,334,994 is disclosed as containing 33% and 3% respectively of SAPO-5. Unfortunately, whereas CHA framework type materials have channels defined by six-membered rings of tetrahedrally coordinated atoms and a pore size of about 0.38 nm, AFI materials have twelve-membered rings channels and a pore size of about 0.73 nm. It has therefore generally been understood that CHA framework type materials used in oxygenate conversion processes should be completely free of AFI framework type impurities.

For example, the article entitled “Selective formation of SAPO-5 and SAPO-34 molecular sieves with microwave radiation and hydrothermal heating” by Sung Hwa Jhung et al, Microporous and Mesoporous Materials 64, (2003), pages 33-39 discloses that, although the CHA and AFI framework types compete, SAPO-34 and SAPO-5 molecular sieves can be selectively formed using hydrothermal heating and microwave radiation, respectively, of the same synthesis gel irrespective of the acidity or type of template, such as triethylamine and N,N,N′,N′-tetraethylethylene diamine.

Unexpectedly, it has now been found that molecular sieves composed at least partly of the CHA framework type and containing up to 2.5 wt %, such as up to 1.5 wt %, preferably up to 0.75 wt % of AFI framework type material can be used in the catalytic conversion of oxygenates, such as methanol, with minimal loss in the selectivity to ethylene and propylene and minimal increase in the production of C₄+ hydrocarbons. In addition, it has been found that X-ray diffraction analysis, and in particular comparison of the area under the X-ray diffraction peak centered at a two-theta value of 7.3° with the area under the X-ray diffraction peak centered at a two-theta value of 9.4°, can be used to determine the amount of AFI framework type impurity phase in a CHA framework type-containing molecular sieve.

U.S. Pat. No. 6,531,639 discloses a method of making an olefin product from an oxygenate-containing feedstock by contacting the feedstock with a non-zeolite catalyst at an oxygenate partial pressure of greater than 20 psia, a weight hourly space velocity of greater than 2 hr⁻¹, an average gas superficial velocity of greater than 1 meter per second, and an oxygenate proportion index of at least 0.5. The catalyst employed is a silicoaluminophosphate (SAPO) molecular sieve selected from SAPO-5, SAPO-8, SAPO- 11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47, SAPO-56, metal-containing forms, mixtures and intergrowths thereof. In addition, further olefin-forming molecular sieve materials can be included as a part of the SAPO catalyst composition or as separate molecular sieve catalysts in admixture with the SAPO catalyst if desired. Examples of suitable small pore molecular sieves are said to include AEI, AFT, APC, ATN, ATT, ATV, AWW, BIK, CAS, CHA, CHI, DAC, DDR, EDI, ERI, GOO, KFI, LEV, LOV, LTA, MON, PAU, PHI, RHO, ROG, and THO structure type materials, whereas examples of suitable medium pore molecular sieves are said to include MFI, MEL, MTW, EUO, MTT, HEU, FER, AFO, AEL and TON structure type materials.

### SUMMARY

In one aspect, the invention resides in a catalyst composition for use in the conversion of oxygenates to olefins, the catalyst composition comprising a first molecular sieve comprising a CHA framework type material and a second molecular sieve comprising an AFI framework type material, wherein said second molecular sieve is present in an amount up to 2.5 % by weight of said first molecular sieve.

Conveniently, said second molecular sieve is present in an amount up to 1.5 %, preferably up to 0.75%, and most preferably up to 0.5% of by weight of said first molecular sieve.

Conveniently, the first molecular sieve comprises a silicoaluminophosphate.

In one embodiment, the first molecular sieve comprises at least one intergrown form of AEI and CHA framework type materials, and in particular at least one intergrown form having an AEI/CHA ratio of from about 5/95 to 40/60, for example from about 10/90 to about 30/70, such as from about 15/85 to about 20/80, as determined by DIFFaX analysis. In a further embodiment, the silicoaluminophosphate molecular sieve comprises first and second intergrown forms each of an AEI framework type material and a CHA framework type material, the first intergrown form having an AEI/CHA ratio of from about 5/95 to about 40/60 as determined by DIFFaX analysis, and the second intergrown from having a different AEI/CHA ratio from said first intergrown form, such as an AEI/CHA ratio of about 50/50 as determined by DIFFaX analysis.

Conveniently, said second molecular sieve comprises an aluminophosphate or a silicoaluminophosphate, such as ALPO-5, SAPO-5 or a substituted form thereof.

In a further aspect, the invention resides in a method of producing a catalyst composition for use in the conversion of oxygenates to olefins, the method comprising:

(a) preparing a synthesis mixture suitable for producing a first molecular sieve comprising a CHA framework material;

(b) crystallizing said synthesis mixture;

(c) recovering from said synthesis mixture a crystalline composition comprising said first molecular sieve and a second molecular sieve comprising an AFI framework type material; and

(d) preparing a catalyst from the crystalline composition recovered in (c), wherein said catalyst comprises said first molecular sieve and said second molecular sieve and wherein said second molecular sieve is present in an amount up to 0.75% by weight of said first molecular sieve.

Conveniently, said crystalline composition recovered in (c) comprises said second molecular sieve in an amount up to 0.75% by weight of said first molecular sieve.

Alternatively, said crystalline composition recovered in (c) comprises said second molecular sieve in an amount in excess of 0.75% by weight of said first molecular sieve and the preparing (d) includes mixing said crystalline composition with additional first molecular sieve to reduce the amount of said second molecular sieve to 0.75% by weight or less of said first molecular sieve.

In yet a further aspect, the invention resides in a method of producing a catalyst for use in the conversion of oxygenates to olefins, the method comprising mixing a first molecular sieve composition with a second molecular sieve composition to produce a third molecular sieve composition, wherein

(a) the first molecular sieve composition comprises a first molecular sieve and a second molecular sieve, said first molecular sieve comprising a CHA framework type material, and said second molecular sieve comprising an AFI framework type material, said second molecular sieve being present in an amount in excess of 0.75 wt.% of the first molecular sieve;

(b) the second molecular sieve composition comprises a molecular sieve comprising a CHA framework type material; and

(c) the ratio of the first and second molecular sieve compositions are such that the third molecular sieve composition contains up to 0.75% of the second molecular sieve by weight of molecular sieve comprising a CHA framework type material.

In still a further aspect, the invention resides in a process for converting an oxygenate-containing feedstock to a product comprising olefins, the process comprising contacting the feedstock under oxygenate to olefin conversion conditions with a catalyst composition comprising a first molecular sieve comprising a CHA framework type material and a second molecular sieve comprising an AFI framework type material, wherein said second molecular sieve is present in an amount up to 0.75 wt% by weight of said first molecular sieve.

### BRIEF DESCRIPTION OF THE DRAWINGS

FIGS. 1a and 1b are DIFFaX simulated diffraction patterns for intergrown AEI/CHA phases having varying AEI/CHA ratios.

FIG. 2 is a graph showing the correlation between the weight of SAPO-5 added to the mixtures of Example 2 and the SAPO-5 content of the mixtures as determined by X-ray analysis.

FIG. 3 is a graph plotting the prime olefin selectivity (POS) and the C₄+ selectivity with SAPO-5 content of the mixtures of Example 2 when used in the conversion of methanol to olefins.

### DETAILED DESCRIPTION OF THE EMBODIMENTS

The present invention relates to a process for converting an oxygenate-containing feedstock, such as methanol, to a product comprising olefins, such as ethylene and propylene, in the presence of a catalyst composition comprising a synthetic crystalline molecular sieve, especially a silicoaluminophosphate molecular sieve, comprising a CHA framework type material. As used herein, the term “molecular sieve comprising a CHA framework type material” is intended to mean that the molecular sieve can be a regular ordered molecular sieve having the CHA framework type, such as SAPO- 34, or can include one or more intergrowths of a CHA framework type material with other framework type materials, such as an AEI framework type material.

It is known that the synthesis of molecular sieves containing CHA framework type materials frequently produces impurity phases and, in particular, that the production of AFI framework type materials, such as SAPO-5, often competes with that of CHA framework type materials. The invention is based on the unexpected finding that the presence of small amounts (up to 2.5 wt %, preferably up to 1.5 wt %, most preferably up to 0.75 wt%) of AFI impurity phase in a CHA framework type molecular sieve does not significantly reduce the selectivity of the CHA framework type molecular sieve to ethylene and propylene when used as an oxygenate conversion catalyst or result in a significant increase in the production of C₄+ hydrocarbons. This is an important result since it means that the conditions used in the synthesis of CHA framework type materials for use in oxygenate conversion catalysts need not be so closely controlled as to avoid all contamination by AFI impurity phases. As a result the synthesis process can be significantly simplified.

In one embodiment, the CHA framework type molecular sieve employed in the process of the invention is a regular ordered molecular sieve and in particular is a silicoaluminophosphate, especially SAPO-34. Regular crystalline solids are built from structurally invariant building units, called Periodic Building Units, and are periodically ordered in three dimensions. For CHA framework type materials, the Periodic Building Unit is a double six ring layer. There are two types of layers “a” and “b”, which are topologically identical except “b” is the mirror image of “a”. When layers of the same type stack on top of one another, i.e . . . aaa . . . or . . . bbb . . . , the framework type CHA is generated. When layers “a” and “b” alternate, e.g. . . . abab . . . , a different framework type, namely AEI, is generated.

SAPO-34 is a well known material that, as described in U.S. Pat. No. 4,440,871, incorporated herein by reference, can be synthesized from an aqueous reaction mixture containing sources of silicon (e.g., a silica sol), aluminum (e.g., hydrated aluminum oxide), and phosphorus (e.g., orthophosphoric acid), and an organic directing agent, for example tetraethylammonium hydroxide (TEAOH), isopropylamine or di-n-propylamine. Other known directing agents for SAPO-34 include triethylamine, cyclohexylamine, 1-methylamidazole, morpholine, pyridine, piperidine, diethylethanolamine, and N,N,N′,N′-tetraethylethylene diamine. Synthesis of the SAPO-34 typically involves hydrothermal treatment of the synthesis mixture at a temperature within the range of about 100° to 250° C. for a time of from 1 to 200 hours. The synthesis also tends to produce an AFI framework type impurity phase, such as SAPO-5, ALPO- 5 or a substituted form thereof, together with the desired SAPO-34. However, according to the invention, it is found that up to 2.5 wt%, conveniently up to 1.5 wt%, preferably up to 0.75 wt%, such as up to 0.5 wt%, of AFI impurity can be present in the SAPO-34 without significant impact on the prime olefin selectivity or the C₄+ make in the conversion of methanol to olefins.

Structurally disordered molecular sieves are also known and show periodic ordering in dimensions less than three, i.e. in two, one or zero dimensions. This phenomenon is called stacking disorder of structurally invariant Periodic Building Units. Intergrown molecular sieve phases are disordered planar intergrowths of molecular sieve frameworks. Reference is directed to the “Catalog of Disordered Zeolite Structures”, 2000 Edition, published by the Structure Commission of the International Zeolite Association and to the “Collection of Simulated XRD Powder Patterns for Zeolites”, M. M. J. Treacy and J. B. Higgins, 2001 Edition, published on behalf of the Structure Commission of the International Zeolite Association for a detailed explanation of intergrown molecular sieve phases.

Thus in another embodiment, the CHA framework type molecular sieve employed in the process of the invention comprises at least one intergrowth of a CHA framework type molecular sieve with another framework type material, particularly an AEI framework type material. In the case of intergrown materials, the interpretation of X-ray diffraction patterns requires an ability to simulate the effects of stacking disorder. DIFFaX is a computer program based on a mathematical model for calculating intensities from crystals containing planar faults (see M. M. J. Tracey et al., Proceedings of the Royal Chemical Society, London, A [1991], Vol. 433, pp. 499-520). DIFFaX is the simulation program selected by and available from the International Zeolite Association to simulate the XRD powder patterns for intergrown phases of zeolites (see “Collection of Simulated XRD Powder Patterns for Zeolites” by M. M. J. Treacy and J. B. Higgins, 2001, Fourth Edition, published on behalf of the Structure Commission of the International Zeolite Association). It has also been used to theoretically study intergrown phases of AEI, CHA and KFI, as reported by K. P. Lillerud et al. in “Studies in Surface Science and Catalysis”, 1994, Vol. 84, pp. 543-550.

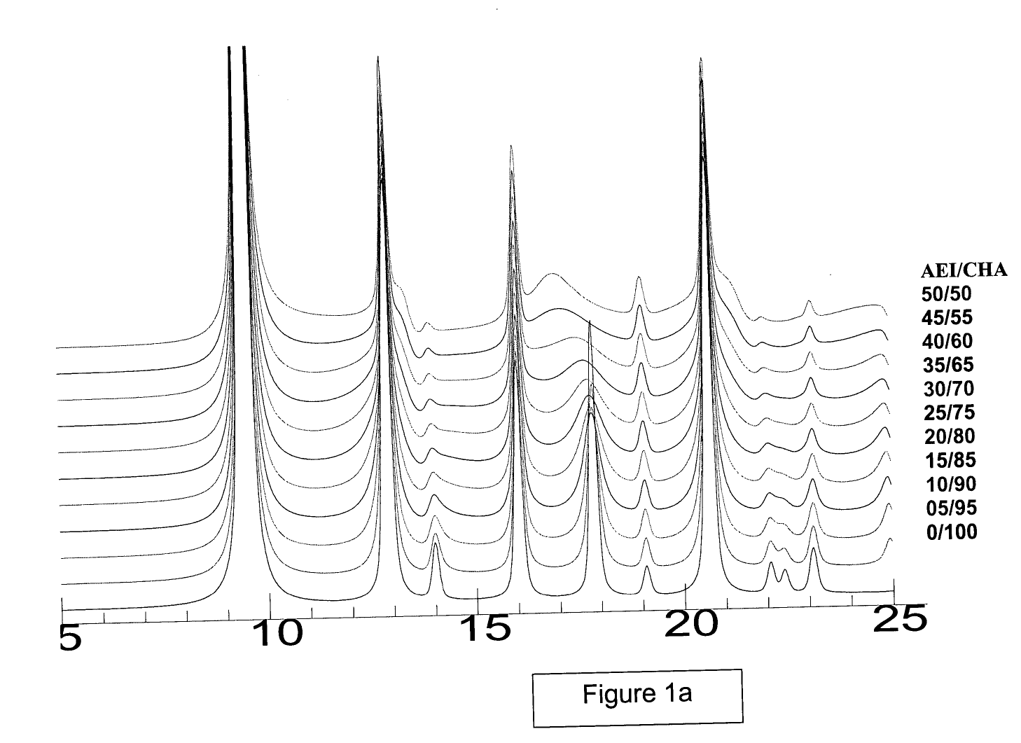

FIGS. 1a and 1b show the simulated diffraction patterns obtained for intergrowths of a CHA framework type molecular sieve with an AEI framework type molecular sieve having various AEI/CHA ratios. FIG. 1a shows the diffraction patterns in the 15 to 35 (2θ) range simulated by DIFFaX for intergrown phases with AEI/CHA ratios of 0/100 (CHA end-member), 10/90 (AEI/CHA=0.11), 20/80 (AEI/CHA=0.25), 30/70 (AEI/CHA=0.41), 40/60 (AEI/CHA=0.67), 50/50 (AEI/CHA=1.00) and 60/40 (AEI/CHA=1.50). FIG. 1b shows the diffraction patterns in the range of 5 to 20 (2θ) simulated by DIFFaX for intergrown phases with AEI/CHA ratios of 0/100 (CHA end-member), 10/90 (AEI/CHA=0.11), 20/80 (AEI/CHA=0,25), 50/50 (AEI/CHA=1,0), 70/30 (AEI/CHA=2.33), 80/20 (AEI/CHA=4.0), 100/0 (AEI end-member). All XRD diffraction patterns are normalized to the highest peak of the entire set of simulated patterns, i.e. the peak at about 9.5 degrees 2θ for pure CHA (AEI/CHA ratio of 0/100). Such normalization of intensity values allows a quantitative determination of mixtures of intergrowths.

As the ratio of AEI increases relative to CHA in the intergrown phase, one can observe a decrease in intensity of certain peaks, for example, the peak at about 2θ=25.0 and an increase in intensity of other peaks, for example the peak at about 2θ=17.05 and the shoulder at 2θ=21.2. Intergrown phases with AEI/CHA ratios of 50/50 and above (AEI/CHA≧1.0) show a broad feature centered at about 16.9 (2θ).

In a preferred embodiment, the CHA framework type molecular sieve employed in the process of the invention is a silicoaluminophosphate comprising at least one intergrowth of a CHA framework type and an AEI framework type, wherein said at least one intergrowth has an AEI/CHA ratio of from about 5/95 to about 40/60, for example from about 10/90 to about 30/70, such as from about 15/85 to about 20/80, as determined by DIFFaX analysis. Such a CHA-rich intergrowth is characterized by a powder XRD diffraction pattern (obtained from a sample after calcination and without rehydration after calcination) having at least the reflections in the 5 to 25 (2θ) range as shown in Table below:

TABLE 1
2θ (CuKα)
9.3-9.6
12.7-13.0
13.8-14.0
15.9-16.1
17.7-18.1
18.9-19.1
20.5-20.7
23.7-24.0

The X-ray diffraction data referred to herein are collected with a SCINTAG X2 X-Ray Powder Diffractometer (Scintag Inc., USA), using copper K-alpha radiation. The diffraction data are recorded by step-scanning at 0.02 degrees of two-theta, where theta is the Bragg angle, and a counting time of 1 second for each step. Prior to recording of each experimental X-ray diffraction pattern, the sample must be in the anhydrous state and free of any template used in its synthesis, since the simulated patterns are calculated using only framework atoms, not extra-framework material such as water or template in the cavities. Given the sensitivity of silicoaluminophosphate materials to water at recording temperatures, the molecular sieve samples are calcined after preparation and kept moisture-free according to the following procedure.

About 2 grams of each molecular sieve sample are heated in an oven from room temperature under a flow of nitrogen at a rate of 3° C./minute to 200° C. and, while retaining the nitrogen flow, the sample is held at 200° C. for 30 minutes and the temperature of the oven is then raised at a rate of 2° C./minute to 650° C. The sample is then retained at 650° C. for 8 hours, the first 5 hours being under nitrogen and the final 3 hours being under air. The oven is then cooled to 200° C. at 30° C./minute and, when the XRD pattern is to be recorded, the sample is transferred from the oven directly to a sample holder and covered with Mylar foil to prevent rehydration. Recording under the same conditions immediately after removal of the Mylar foil will also provide a diffraction pattern suitable for use in DIFFaX analysis.

In an alternative embodiment, the CHA framework type molecular sieve employed in the process of the invention is a silicoaluminophosphate comprising a plurality of intergrown forms of the CHA and AEI framework types, typically with a first intergrown form having an AEI/CHA ratio of from about 5/95 to about 40/60, as determined by DIFFaX analysis, and a second intergrown form having a different AEI/CHA ratio from said first intergrown form. The second intergrown form typically has an AEI/CHA ratio of about 50/50, as determined by DIFFaX analysis, in which case the XRD diffraction pattern exhibits a broad feature centered at about 16.9 (2θ) in addition to the reflection peaks listed in Table 1.

Preferably, where the CHA framework type silicoaluminophosphate comprises at least one intergrowth of CHA and AEI framework type molecular sieves, the CHA molecular sieve is SAPO-34 and the AEI molecular sieve is selected from SAPO-18, ALPO-18 and mixtures thereof. In addition, the intergrown silicoaluminophosphate preferably has a framework silica to alumina molar ratio (Si/Al₂) greater than 0.16 and less than 0.19, such as from about 0.165 to about 0.185, for example about 0.18. The framework silica to alumina molar ratio is conveniently determined by NMR analysis.

Silicoaluminophosphate molecular sieves comprising CHA/AEI intergrowths may conveniently be prepared by a process that comprises

a) combining reactive sources of silicon, phosphorus and aluminum with an organic structure directing agent (template) to form a mixture having a molar composition within the following ranges:
P₂O₅:Al₂O₃ from about 0.6 to about 1.2,
SiO₂:Al₂O₃ from about 0.005 to about 0.35,
H₂O:Al₂O₃ from about 10 to about 50;

b) mixing and heating the mixture (a) continuously to a crystallization temperature, such as between about 100° C. and about 250° C., typically between about 140° C. and about 180° C., preferably between about 150° C. and about 170° C.;

c) maintaining the mixture at the crystallization for a period of time of from 2 to 150 hours; such as from about 5 to about 100 hours, for example from about 10 to about 50 hours; and

(d) recovering the desired molecular sieve.

The reactive source of silicon used in the above mixture may be a silicate, e.g., fumed silica, such as Aerosil (available from Degussa), a tetraalkyl orthosilicate, or an aqueous colloidal suspension of silica, for example that sold by E. I. du Pont de Nemours under the tradename Ludox. The reactive source of phosphorus used in the above mixture is conveniently phosphoric acid. Examples of suitable reactive aluminum sources include hydrated aluminum oxides such as boehmite and pseudoboehmite. Preferably, pseudoboehmite is used. The organic structure directing agent conveniently includes a tetraethyl ammonium compound, such as tetraethyl ammonium hydroxide (TEAOH), tetraethyl ammonium phosphate, tetraethyl ammonium fluoride, tetraethyl ammonium bromide, tetraethyl ammonium chloride or tetraethyl ammonium acetate. Typically, the directing agent includes tetraethyl ammonium hydroxide. In some cases, more than one organic structure directing agent may be employed, such as a combination of a tetraethyl ammonium compound and dipropylamine.

The crystalline product recovered in step (d) above will tend to contain an AFI framework type impurity phase, such as SAPO-5, ALPO-5 or a substituted form thereof, in addition the desired CHA/AEI intergrowth. However, when the crystalline product is used in a catalyst composition for the conversion of methanol to olefins, it is found that there is no significant impact on the prime olefin selectivity or the C₄+ make of the catalyst composition provided the AFI impurity phase is no more than 2.5 wt %, especially no more than 1.5 wt %, preferably no more than 0.75 wt%, such as no more than 0.5 wt%, of the intergrowth.

As a result of the synthesis process, the recovered crystalline product, whether SAPO-34 or CHA/AEI intergrowth, contains within its pores at least a portion of the organic directing agent used in the synthesis. In a preferred embodiment, activation is performed in such a manner that the organic directing agent is removed from the molecular sieve, leaving active catalytic sites within the microporous channels of the molecular sieve open for contact with a feedstock. The activation process is typically accomplished by calcining, or essentially heating the molecular sieve comprising the template at a temperature of from about 200° C. to about 800° C. in the presence of an oxygen-containing gas. In some cases, it may be desirable to heat the molecular sieve in an environment having a low or zero oxygen concentration. This type of process can be used for partial or complete removal of the organic directing agent from the intracrystalline pore system. In other cases, particularly with smaller organic directing agents, complete or partial removal from the sieve can be accomplished by conventional desorption processes.

Before use in the process of the invention, the crystalline product will normally be formulated into a catalyst composition by combination with other materials, such as binders and/or matrix materials, which provide additional hardness or catalytic activity to the finished catalyst.

Materials which can be blended with the intergrown crystalline material of the invention can be various inert or catalytically active materials. These materials include compositions such as kaolin and other clays, various forms of rare earth metals, other non-zeolite catalyst components, zeolite catalyst components, alumina or alumina sol, titania, zirconia, quartz, silica or silica sol, and mixtures thereof. These components are also effective in reducing overall catalyst cost, acting as a thermal sink to assist in heat shielding the catalyst during regeneration, densifying the catalyst and increasing catalyst strength. When blended with such components, the amount of intergrown crystalline material contained in the final catalyst product ranges from 10 to 90 weight percent of the total catalyst, preferably 20 to 80 weight percent of the total catalyst.

The resultant catalyst composition is found to be effective in the conversion of oxygenates to olefins, despite the presence of small quantities of AFI phase impurity that may be present in addition to the desired SAPO-34 or CHA/AEI intergrowth.

In practice, the synthesis process to produce the desired CHA- containing molecular sieve (SAPO-34 or CHA/AEI intergrowth) can be controlled to ensure that the amount of AFI impurity phase produced during the synthesis is no more than 2.5 % by wegith, preferably no more than 1.5 wt %, such as no more than 0.75 % by weight of the CHA-containing molecular sieve. Alternatively, if the synthesis product contains more than the desired amount of the AFI impurity phase by weight of the CHA-containing molecular sieve, additional CHA-containing molecular sieve can be mixed therewith to reduce the overall content of AFI impurity phase in the final catalyst composition to the desired level with respect to the CHA-containing molecular sieve. The mixing can be effected on the as-synthesized crystalline product, after removal of the organic directing from the as-synthesized crystalline product, or after combining the crystalline product with a binder and/or matrix.

As used herein, the term “oxygenates” is defined to include, but is not necessarily limited to aliphatic alcohols, ethers, carbonyl compounds (aldehydes, ketones, carboxylic acids, carbonates, and the like), and also compounds containing hetero-atoms, such as, halides, mercaptans, sulfides, amines, and mixtures thereof. The aliphatic moiety will normally contain from about 1 to about 10 carbon atoms, such as from about 1 to about 4 carbon atoms.

Representative oxygenates include lower straight chain or branched aliphatic alcohols, their unsaturated counterparts, and their nitrogen, halogen and sulfur analogues. Examples of suitable oxygenate compounds include methanol; ethanol; n-propanol; isopropanol; C₄-C₁₀ alcohols; methyl ethyl ether; dimethyl ether; diethyl ether; di-isopropyl ether; methyl mercaptan; methyl sulfide; methyl amine; ethyl mercaptan; di-ethyl sulfide; di-ethyl amine; ethyl chloride; formaldehyde; di-methyl carbonate; di-methyl ketone; acetic acid; n-alkyl amines, n-alkyl halides, n-alkyl sulfides having n-alkyl groups of comprising the range of from about 3 to about 10 carbon atoms; and mixtures thereof. Particularly suitable oxygenate compounds are methanol, dimethyl ether, or mixtures thereof, most preferably methanol. As used herein, the term “oxygenate” designates only the organic material used as the feed. The total charge of feed to the reaction zone may contain additional compounds, such as diluents.

In the present oxygenate conversion process, a feedstock comprising an organic oxygenate, optionally with one or more diluents, is contacted in the vapor phase in a reaction zone with a catalyst comprising the molecular sieve of the present invention at effective process conditions so as to produce the desired olefins. Alternatively, the process may be carried out in a liquid or a mixed vapor/liquid phase. When the process is carried out in the liquid phase or a mixed vapor/liquid phase, different conversion rates and selectivities of feedstock-to-product may result depending upon the catalyst and the reaction conditions.

When present, the diluent(s) is generally non-reactive to the feedstock or molecular sieve catalyst composition and is typically used to reduce the concentration of the oxygenate in the feedstock. Non-limiting examples of suitable diluents include helium, argon, nitrogen, carbon monoxide, carbon dioxide, water, essentially non-reactive paraffins (especially alkanes such as methane, ethane, and propane), essentially non-reactive aromatic compounds, and mixtures thereof. The most preferred diluents are water and nitrogen, with water being particularly preferred. Diluent(s) may comprise from about 1 mol % to about 99 mol % of the total feed mixture.

The temperature employed in the oxygenate conversion process may vary over a wide range, such as from about 200° C. to about 1000° C., for example from about 250° C. to about 800° C., including from about 250° C. to about 750° C., conveniently from about 300° C. to about 650° C., typically from about 350° C. to about 600° C. and particularly from about 400° C. to about 600° C.

Light olefin products will form, although not necessarily in optimum amounts, at a wide range of pressures, including but not limited to autogenous pressures and pressures in the range of from about 0.1 kPa to about 10 MPa. Conveniently, the pressure is in the range of from about 7 kPa to about 5 MPa, such as in the range of from about 50 kPa to about 1 MPa. The foregoing pressures are exclusive of diluent, if any is present, and refer to the partial pressure of the feedstock as it relates to oxygenate compounds and/or mixtures thereof. Lower and upper extremes of pressure may adversely affect selectivity, conversion, coking rate, and/or reaction rate; however, light olefins such as ethylene still may form.

The process should be continued for a period of time sufficient to produce the desired olefin products. The reaction time may vary from tenths of seconds to a number of hours. The reaction time is largely determined by the reaction temperature, the pressure, the catalyst selected, the weight hourly space velocity, the phase (liquid or vapor) and the selected process design characteristics.

A wide range of weight hourly space velocities (WHSV) for the feedstock will function in the present process. WHSV is defined as weight of feed (excluding diluent) per hour per weight of a total reaction volume of molecular sieve catalyst (excluding inerts and/or fillers). The WHSV generally should be in the range of from about 0.01 hr⁻¹ to about 500 hr⁻¹, such as in the range of from about 0.5 hr⁻¹ to about 300 hr^{-1,} for example in the range of from about 0.1 hr⁻¹ to about 200 hr⁻¹.

A practical embodiment of a reactor system for the oxygenate conversion process is a circulating fluid bed reactor with continuous regeneration, similar to a modem fluid catalytic cracker. Fixed beds are generally not preferred for the process because oxygenate to olefin conversion is a highly exothermic process which requires several stages with intercoolers or other cooling devices. The reaction also results in a high pressure drop due to the production of low pressure, low density gas.

Because the catalyst must be regenerated frequently, the reactor should allow easy removal of a portion of the catalyst to a regenerator, where the catalyst is subjected to a regeneration medium, such as a gas comprising oxygen, for example air, to bum off coke from the catalyst, which restores the catalyst activity. The conditions of temperature, oxygen partial pressure, and residence time in the regenerator should be selected to achieve a coke content on regenerated catalyst of less than about 0.5 wt %. At least a portion of the regenerated catalyst should be returned to the reactor.

Using the various oxygenate feedstocks discussed above, particularly a feedstock containing methanol, the catalyst composition of the invention is effective to convert the feedstock primarily into one or more olefin(s). The olefin(s) produced typically have from 2 to 30 carbon atoms, preferably 2 to 8 carbon atoms, more preferably 2 to 6 carbon atoms, still more preferably 2 to 4 carbons atoms, and most preferably are ethylene and/or propylene. The resultant olefins can be separated from the oxygenate conversion product for sale or can be fed to a downstream process for converting the olefins to, for example, polymers.

The invention will now be more particularly described with reference to the following Examples.

In the examples, DIFFaX analysis was used to determine the AEI/CHA ratio of the molecular sieves. Simulated powder XRD diffraction patterns for varying ratios of AEI/CHA were generated using the DIFFaX program available from the International Zeolite Association (see also M. M. J. Tracey et al., Proceedings of the Royal Chemical Society, London, A (1991), Vol. 433, pp. 499-520 “Collection of Simulated XRD Powder Patterns for Zeolites” by M. M. J. Treacy and J. B. Higgins, 2001, Fourth Edition, published on behalf of the Structure Commission of the International Zeolite Association). The DIFFaX input file used to simulate the XRD diffraction patterns is given in Table 2 of U.S. Patent Application Publication No. 2002/0165089, incorporated herein by reference. In order to obtain best fitting between the DIFFaX simulated patterns and the experimental patterns, two sets of simulated XRD patterns were generated using a line broadening of 0.009 (as described in U.S. Patent Application No. 2002/0165089) and a line broadening of 0.04 (FIGS. 1a and 1b). The simulated diffraction patterns were then compared with the experimental powder XRD diffraction patterns. In this respect, a very sensitive range is the 15 to19.5 2θ range.

### EXAMPLE 1

A mixture of 5017 kg of phosphoric acid (85% in water), 4064 kg of demineralized water and 9157 kg of tetraethylammonium hydroxide solution (35% in water, Sachem) was prepared in a mixing tank and, after initiating stirring of the mixture, 392 kg Ludox AS 40 (40% silica) was added followed by 2873 kg of alumina (Condea Pural SB-1) and 422 kg of rinse water. The composition of the final synthesis mixture in terms of molar ratios was as follows:
0.12SiO₂/Al₂O₃/P₂O₅/TEAOH/34H₂O

The mixture was transferred to a stainless steel reactor and heated at 20° C./hour to 165° C. while stirring. The reactor was kept at 165° C. for 60 hours. After cooling to room temperature, the slurry was washed and dried and an X-ray diffraction pattern of the crystalline product was taken after the calcination procedure described above. Using this diffraction pattern, DIFFaX analysis was conducted and showed the crystalline product to contain a single AEI/CHA intergrowth having an AEI/CHA ratio of 26/74. No SAPO-5 was detected in the crystalline product. The framework silica to alumina molar ratio (Si/Al₂) of the crystalline product was found to be 0.14.

### EXAMPLE 2

Physical mixtures of the AEI/CHA intergrowth produced in Example 1 with SAPO-5 having a silica to alumina molar ratio (Si/Al₂) of 0.05 were produced in which the SAPO-5 content varied between of 0.5 to 6.5 wt% of the mixture.

Samples of the mixtures were then calcined at 650° C. for 8 hours, the first 5 hours being under nitrogen and the final 3 hours being under air and subjected to X-ray analysis in which each sample was transferred hot (˜170° C.) to an XRD sample cup and covered with Mylar foil to prevent rehydration. XRD patterns were recorded over a 2-theta range of 5 to 35° and the amount of SAPO-5 in the samples was calculated according to the equation:

$$\% SAPO \text{-} 5 = \frac{area of SAPO \text{-} 5 peak centered at 2 \text{-} theta of 7.3}{area of AEI \text{/} CHA peak centered at 2 \text{-} theta of 9.4}$$

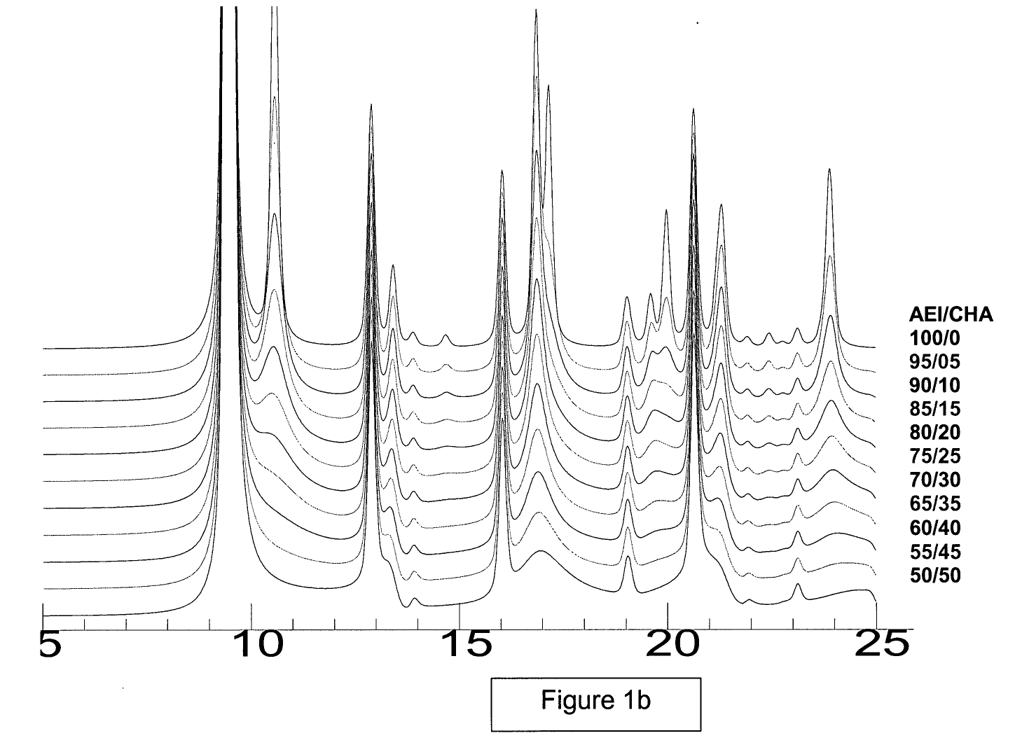

The areas of the peaks were determined by peak fitting in the range 6<2θ<11 using Voigt (area) functions, with the areas being background corrected. The results are given in FIG. 2 and show excellent correlation between the actual and measured SAPO-5 values, demonstrating that X-ray diffraction can be used to determine SAPO-5 levels as low as 0.5 wt% in SAPO-34 or AEI/CHA intergrowths.

### EXAMPLE 3

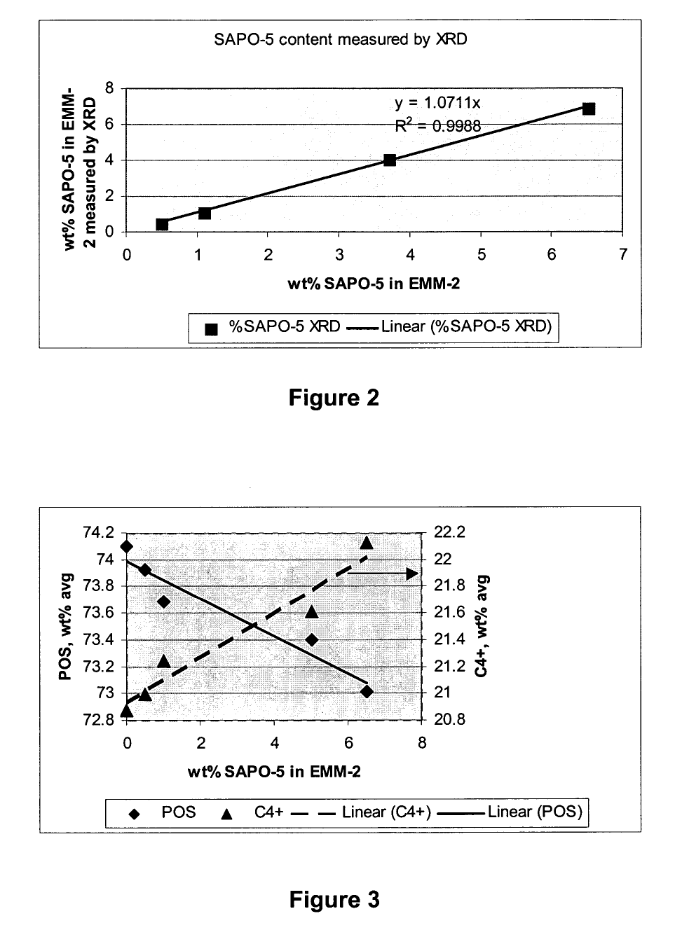

The calcined mixtures produced in Example 2 were evaluated for MTO performance in a fixed bed reactor, equipped with on-line gas chromatography, at 475° C., 100 WHSV and 25 psig (273 kPa) methanol partial pressure. The performance of these mixtures is compared with that of the AEI/CHA intergrowth of Example 1 in FIG. 3, in which prime olefin selectivity (POS) equates to the total selectivity of ethylene and propylene in the product. It will be seen from FIG. 3 that the presence of 0.5 wt% SAPO-5 in the AEI/CHA intergrowth had no significant affect on the POS or C₄+ selectivity of the intergrowth.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

We claim:

1. A catalyst composition for use in the conversion of oxygenates to olefins, the catalyst composition comprising a first molecular sieve comprising a CHA framework type material and a second molecular sieve comprising an AFI framework type material, wherein said second molecular sieve is present in an amount up to 2.5% by weight of said first molecular sieve.

2. The catalyst composition of claim 1, wherein said second molecular sieve is present in an amount up to 1.5% by weight of said first molecular sieve.

3. The catalyst composition of claim 1, wherein said second molecular sieve is present in an amount up to 1.0% by weight of said first molecular sieve.

4. The catalyst composition of claim 1, wherein said second molecular sieve is present in an amount up to 0.75% by weight of said first molecular sieve.

5. The catalyst composition of claim 1, wherein said second molecular sieve is present in an amount up to 0.5% by weight of said first molecular sieve.

6. The catalyst composition of claim 1, wherein said first molecular sieve comprises a silicoaluminophosphate.

7. The catalyst composition of claim 1, wherein said first molecular sieve comprises at least one intergrown form of a CHA framework type material and an AEI framework type material.

8. The catalyst composition of claim 1, wherein said first molecular sieve comprises at least one intergrown form having an AEI/CHA ratio of from about 5/95 to about 40/60 as determined by DIFFaX.

9. The catalyst composition of claim 1, wherein said first molecular sieve comprises at least one intergrown form having an AEI/CHA ratio of from about 10/90 to about 30/70 as determined by DIFFaX.

10. The catalyst composition of claim 1, wherein said first molecular sieve comprises at least one intergrown form having an AEI/CHA ratio of from about 15/95 to about 20/80 as determined by DIFFaX.

11. The catalyst composition of claim 8, wherein said first molecular sieve has an X-ray diffraction pattern comprising at least one reflection peak in each of the following ranges in the 5 to 25 (28) range:
2θ (CuKα)
9.3-9.6
12.7-13.0
13.8-14.0
15.9-16.1
17.7-18.1
18.9-19.1
20.5-20.7
23.7-24.0.

12. The catalyst composition of claim 1, wherein said first molecular sieve comprises first and second intergrown forms each of an AEI framework type material and a CHA framework type material.

13. The catalyst composition of claim 12, wherein said first intergrown form has an AEI/CHA ratio of from about 5/95 to about 40/60 as determined by DIFFaX analysis and said second intergrown form has a different AEI/CHA ratio from said first intergrown form.

14. The catalyst composition of claim 13, wherein said second intergrown form has an AEI/CHA ratio of about 50/50 as determined by DIFFaX analysis.

15. The catalyst composition of claim 1, wherein said second molecular sieve comprises an aluminophosphate, a silicoaluminophosphate or a substituted form thereof.

16. The catalyst composition of claim 15, wherein said second molecular sieve comprises SAPO-5.

17. The catalyst composition of claim 15, wherein said second molecular sieve comprises ALPO-5.

18. The catalyst composition of claim 1 wherein said amount of said first molecular sieve is determined according to the equation:
A₁/A₂
wherein A₁ is the area under under any X-ray diffraction peak of said composition centered at a two-theta value of about 7.3° and A₂ is the area under any X-ray diffraction peak of said composition centered at a two- theta value of about 9.4°.

19. A method of producing a catalyst for use in the conversion of oxygenates to olefins, the method comprising:
(a) preparing a synthesis mixture suitable for producing a first molecular sieve comprising a CHA framework material;
(b) crystallizing said synthesis mixture;
(c) recovering from said synthesis mixture a crystalline composition comprising said first molecular sieve and a second molecular sieve comprising an AFI framework type material; and
(d) preparing a catalyst from the crystalline composition recovered in (c), wherein said catalyst comprises said first molecular sieve and said second molecular sieve and wherein said second molecular sieve is present in an amount up to 0.75% by weight of said first molecular sieve.

20. The method of claim 19, wherein said crystalline composition recovered in (c) comprises said second molecular sieve in an amount up to 0.75% by weight of said first molecular sieve.

21. The method of claim 19, wherein said crystalline composition recovered in (c) comprises said second molecular sieve in an amount in excess of 0.75% by weight of said first molecular sieve and the preparing (d) includes mixing said crystalline composition with additional first molecular sieve to reduce the amount of said second molecular sieve in said catalyst to 0.75% by weight or less of said first molecular sieve.

22. The method of claim 19, wherein said second molecular sieve is present in said catalyst in an amount up to 0.5% of by weight of said first molecular sieve.

23. A method of producing a catalyst for use in the conversion of oxygenates to olefins, the method comprising mixing a first molecular sieve composition with a second molecular sieve composition to produce a third molecular sieve composition, wherein
(a) the first molecular sieve composition comprises a first molecular sieve and a second molecular sieve, said first molecular sieve comprising a CHA framework type material, and said second molecular sieve comprising an AFI framework type material, said second molecular sieve being present in an amount in excess of 0.75 wt.% of the first molecular sieve;
(b) the second molecular sieve composition comprises a molecular sieve comprising a CHA framework type material; and
(c) the ratio of the first and second molecular sieve compositions being such that the third molecular sieve composition contains up to 0.75% of the second molecular sieve by weight of molecular sieve comprising a CHA framework type material.

24. The method of claim 23, wherein the third molecular sieve composition contains up to 0.5% of the second molecular sieve by weight of molecular sieve comprising a CHA framework type material.

25. The method of claim 23, wherein at least one of the first and second molecular sieve compositions also comprises a matrix and/or binder.

26. A process for converting an oxygenate-containing feedstock to a product comprising olefins, the process comprising contacting the feedstock under oxygenate to olefin conversion conditions with a composition comprising a first molecular sieve comprising a CHA framework type material and a second molecular sieve including a AFI framework type material, wherein said second molecular sieve is present in an amount up to 2.5 wt% of by weight of said first molecular sieve.

27. The process of claim 26, wherein the oxygenate-containing feedstock comprises methanol, dimethyl ether, or mixtures thereof and the product comprises ethylene and propylene.

28. The process of claim 26 and further comprising converting the olefins to polymer.
